(19) United States
(12) Patent Application Publication
(10) Pub. No.: US 2009/0326056 A1
(43) Pub. Date: Dec. 31, 2009

(54) NOVEL ACTIVES AGAINST PROSTATE CARCINOMA

(76) Inventors: Regina Goralczyk, Grenzach-Wyhlen (DE); Ulrich Siler, Grenzach-Wyhlen (DE); Karin Wertz, Rheinfelden (DE)

Correspondence Address: NIXON & VANDERHYE, PC, 901 NORTH GLEBE ROAD, 11TH FLOOR, ARLINGTON, VA, 22203, US

(21) Appl. No.: 12/299,825

(22) PCT Filed: May 15, 2007
(86) PCT No.: PCT/EP2007/004302

§ 371 Date: Jun. 2, 2009

(30) Foreign Application Priority Data
May 15, 2006 (EP) 06009926.4

(51) Int. Cl. A61K 31/352

(52) U.S. Cl. 514/456

(57) ABSTRACT

The present invention relates to the use of a composition comprising lycopene and genistein in the prevention and coadjuvant treatment of prostate carcinoma. The present invention is also directed to the use of a composition comprising lycopene and epigallocatechin gallate in the prevention and coadjuvant treatment of prostate carcinoma. More specifically, the present invention relates to the use of one of these compositions in the primary prevention (i.e., the prophylactic supplementation of healthy subjects) of the onset of prostate carcinoma, in the coadjuvant treatment (i.e. the supplementation accompanying a running therapy of prostate carcinoma) and in the secondary prevention (i.e., the supplementation after a successful therapy for the prevention of relapse) of prostate carcinoma. Furthermore, the present invention is also directed to the reduction/decrease of the PSA plasma level in the blood of subjects to whom either a composition comprising lycopene and genistein or to whom either a composition comprising lycopene and epigallocatechin gallate are administered. Further compounds that may preferably also be present in the composition of the present invention are vitamin E, resveratrol and polyunsaturated fatty acid (derivative)s.

The present invention relates to the use of a composition comprising lycopene and genistein in the prevention and coadjuvant treatment of prostate carcinoma. The present invention is also directed to the use of a composition comprising lycopene and epigallocatechin gallate in the prevention and coadjuvant treatment of prostate carcinoma. More specifically, the present invention relates to the use of one of these compositions in the primary prevention (i.e., the prophylactic supplementation of healthy subjects) of the onset of prostate carcinoma, in the coadjuvant treatment (i.e. the supplementation accompanying a running therapy of prostate carcinoma) and in the secondary prevention (i.e., the supplementation after a successful therapy for the prevention of relapse) of prostate carcinoma.

The present invention is also directed to the reduction/decrease of the PSA plasma level in the blood of subjects to whom either a composition comprising lycopene and genistein or to whom either a composition comprising lycopene and epigallocatechin gallate are administered, preferably the present invention is directed to such a reduction/decrease by up to 30%, more preferably by 20-30%. The PSA plasma level is the standard marker for prostate cancer screening and is indicative of prostate size and/or prostatitis. Especially PSA velocity, i.e. the speed by which PSA levels increase is a strong indication of an emerging prostate carcinoma. The total serum PSA may be measured by using a Microparticle Enzyme Immunoassay (Abbot Laboratories, Abbot Park, Ill.). Changes in measured values may be assessed by paired t test with complete data sets. Leukocyte and prostate DNA 80 HdG/dG ratios and apoptotic index and PSA concentrations may be square root or log transformed to overcome skewness.

Prostate cancer represents a severe health problem, especially in the Western world. In 2000, 415,568 of the 542,990 prostate cancer cases diagnosed worldwide were diagnosed in more developed countries, 211,950 of them in North America (see Ferlay, J., Bray, F., Pisani, P. and Parkin, D. M. GLOBOCAN 2000: Cancer Incidence, Mortality and Prevalence Worldwide, Version 1.0, IARC CancerBase No. 5. Lyon, IARCPress, 2001. http://www-dep.iarc.fr/globocan/globocan.html). For 2003, 220,900 cases of prostate cancer (see American Cancer Society (2004) Cancer Facts & Figures 2003, http://www.cancer.org/downloads/STT/CAFF2003PWSecured.pdf) are expected to be diagnosed in the US.

Prostate cancer develops via preneoplastic lesions, called PIN or prostatic intraepithelial neoplasia, which is a premalignant proliferation arising within the prostate. PIN will be identified in up to 16% of men who have had transrectal ultrasound guided prostate biopsies. PIN is most commonly found in the peripheral zone. In PIN the cellular arrangement shows preservation of duct and gland architecture with progressive disruption of the basal cell layer with increasing grades of PIN while invasion of the stroma is lacking. Other histological or biologic changes that have been reported include: loss of neuroendocrine and secretory differentiation, nuclear and nucleolar abnormalities, neovascularity, increased proliferative potential and genetic instability with variation of DNA content. With increasing degrees of PIN an increasing degree of nuclear aberration is seen along with increasing basal cell disruption. PIN has an intact or fragmented basal cell layer, whereas cancer (PC) lacks a basal cell layer. Basal cell specific immunostaining for Cytokeratin is present in PIN but is absent in areas of PC.

PIN is graded from the lowest grade (Grade I) with the least changes to the highest grade (Grade III) with the most severe changes. Most medical papers categorize PIN as either high grade or low grade. High grade PIN and Grade III PIN are used synonymously. PC is associated more with high grade PIN than low grade. PIN appears to predate the appearance of PC by more than 5 years. (Pin definition taken from http://www.prostate-cancer.org/education/preclin/pin.html)

According to the present invention, it has been found that prostate carcinoma and the precursor “prostatic intraepithelial neoplasia” (PIN, high grade PIN (HG-PIN)) can be suppressed or inhibited by the administration of a composition comprising lycopene and genistein or by a composition comprising lycopene and epigallocatechin gallate.

The present invention, therefore, is concerned with the use of a composition comprising lycopene and genistein in the manufacture of a composition for the primary and secondary prevention of prostate cancer and coadjuvant treatment thereof. Alternatively a composition comprising lycopene and epigallocatechin gallate may be used.

“Prevention of prostate carcinoma and the precursor PIN/HG-PIN” in the context of the present invention encompasses also the reduction of the risk of getting prostate carcinoma or the precursor PIN/HG-PIN and the risk of the incidence of prostate carcinoma or the precursor PIN/HG-PIN.

Furthermore, the present invention is concerned with a method of preventing or treating prostate cancer which comprises administering to a subject in need of such treatment for therapy or prophylaxis an effective amount of a composition comprising lycopene and genistein or an effective amount of a composition comprising lycopene and epigallocatechin gallate. Moreover, the present invention is concerned with a method of reducing/decreasing the PSA plasma level in the blood of a subject which comprises administering to said subject in need of such treatment an effective amount of lycopene and genistein or an effective amount of lycopene and epigallocatechin gallate. The present invention is also concerned with certain novel solid galenical formulations comprising such compositions.

In a further and preferred embodiment of the invention, the compositions used may also contain vitamin E and/or resveratrol. Most preferred they contain both, vitamin E and resveratrol.

In another embodiment of the present invention the compositions used may also contain polyunsaturated fatty acids (PUFAs)); as well as their esters, especially their triglycerides.

In a further embodiment of the present invention the compositions used may also contain Vitamin D2 or vitamin D3 or derivatives thereof such as 1α,25-dihydroxyvitamin D3 or 25-hydroxyvitamin D3 or 1α,24R,25-trihydroxyvitamin D3.

In another embodiment of the present invention the compositions used may also contain vitamin C. The term “vitamin C” as used herein includes derivatives thereof which have biological vitamin C activity, e.g. esters and salts, such as sodium ascorbate, sodium ascorbyl phosphate, and ascorbyl palmitate.

In a further embodiment of the invention, the following components are essentially absent in the compositions used:
(a) Astaxanthin ((3S, 3′S)-3,3′-dihydroxy-β,β-carotene-4,4′-dione) and/or one or more isomers and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid;
(b) β-Carotene and/or one or more isomers thereof;
(c) β-Cryptoxanthin ((3R)-β,β-carotene-3-ol) and/or one or more isomers or esters thereof, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid;
(d) Lutein ((3R, 3′R, 6′R)-β,ε, carotene-3,3′-diol) and/or one or more isomers and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid, thereof;
(e) Quercetin (2-(3,4-dihydroxyphenyl)-3,5,7-trihydroxy-4H-1-benzopyrano-4-one) and/or dihydroquercetin and/or one or more derivatives thereof (quercetine glucosides, quercetin glucuronides, quercetine sulphates, methylquercetins (isohamnetin (3′-O-methylquercetin), tamarixetin(4′-O-methylquercetin));
(f) Myricetin and/or one or more derivatives thereof;
(g) Rhizoxin and/or one or more derivatives thereof (palmitoyl rhizoxin);
(h) Silymarin (extract from Silybum marianum) and/or one or more derivatives thereof (silymarin dihemisuccinate sodium salt) and/or one or more of its four main components (silybin [synonymous with silibinin, and sometimes incorrectly called silybinin] and/or isosilybin and/or silydianin and/or silychristin) and/or one or more derivatives thereof (silybin-dihemisuccinate, disilybin, silybin-phosphatidylcholine complex, silybin-phosphate);
(i) Vitamin A and/or one or more derivatives thereof (all-trans retinol or all-trans retinyl acetate or all-trans retinyl palmitate);
(j) Zeaxanthin ((3R, 3′R)-β,β-carotene-3,3′-diol) and/or one or more isomers and stereo-isomers (preferably mesozeaxanthin, 3R,3′S-zeaxanthin) and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid, thereof;
(k) Apigenin and/or one or more derivatives thereof;
(l) Carnosic acid and/or one or more derivatives thereof;
(m) Carnosol and/or one or more derivatives thereof;
(n) Depudecin and/or one or more derivatives thereof;
(o) Eponemycin and/or one or more derivatives thereof;
(p) Dihydroeponemycin and/or one or more derivatives thereof;
(q) Epoxomicin and/or one or more derivatives thereof;
(r) Ergosterol and/or one or more derivatives thereof;
(s) Fisetin and/or one or more derivatives thereof;
(t) Fumagillin and/or one or more derivatives thereof;
(u) Lactacystin and/or one or more derivatives thereof;
(v) Luteolin and/or one or more derivatives thereof;
(w) Motuporamine C and/or one or more derivatives thereof;
(x) Ovalicin and/or one or more derivatives thereof;
(y) Radicicol and/or one or more derivatives thereof;
(z) Curcumin and/or one or more derivatives (demethoxy-curcumin, bis-demethoxycurcumin, sodium curcumionate, bis-demethylcurcumin, tetrahydrocurcumin, diacteylcurcumin, triethylcurcumin) thereof;
(aa) Squalamine and/or one or more derivatives thereof;
(bb) Isoliquiritin, isoliquiritigenin, liquiritigenin and/or one or more derivatives thereof;
(cc) Shark cartilage extract.
(dd) Glucosinolate derivatives (Methylsulfinylalkyl glucosinolates [1-methylsulfinylmethyl glucosinolate, 2-methylsulfinylethyl glucosinolate, 3-methylsulfinylpropyl glucosinolate (glucoiberin), 4-methylsulfinylbutyl glucosinolate (glucoraphanin), 5-methylsulfinylpentyl glucosinolate (glucoalysin), 6-methylsulfinylhexyl glucosinolate, 7-methylsulfinylheptyl glucosinolate, 8-methylsulfinyloctyl glucosinolate, 9-methylsulfinylnonyl glucosinolate, 10-methylsulfinyldodecyl glucosinolate] or allyl glucosinolate (sinigrin) or phenylethyl glucosinolate (gluconasturtiin) or 3-butenyl glucosinolate (gluconapin) or indol-3-ylmethyl glucosinolate (glucobrassicin) or derivatives thereof [N-methoxyindol-3-ylmethyl glucosinolate (neoglucobrassicin), 4-hydroxyindol-3-ylmethyl glucosinolate (4-OH glucobrassicin), 4-methoxyindol-3-ylmethyl glucosinolate (4-CH₃O glucobrassicin)]).
(ee) Isothiocyanate derivatives (Methylsulfinylalkyl isothiocyanate [1-methylsulfinylmethyl isothiocyanate, 2-methylsulfinylethyl isothiocyanate, 3-methylsulfinylpropyl isothiocyanate, 4-methylsulfinylbutyl isothiocyanate (sulforaphane), 5-methylsulfinylpentyl isothiocyanate, 6-methylsulfinylhexyl isothiocyanate (6-HITC), 7-methylsulfinylheptyl isothiocyanate, 8-methylsulfinyloctyl isothiocyanate, 9-methylsulfinylnonyl isothiocyanate, 10-methylsulfinyldodecyl isothiocyanate] or allyl isothiocyanate or phenylethyl isothiocyanate (PEITC) or 3-butenyl isothiocyanate or indol-3-ylmethylisothiocyanate or derivatives thereof (N-methoxy indol-3-ylmethylisothiocyanate, 4-hydroxy indol-3-ylmethylisothiocyanate, 4-methoxy indol-3-ylmethylisothiocyanate) or 3-indolmethanol (indol-3-carbinol, 13C).

“Essentially absent” in the context of the present invention means that the amount in the compositions used according to the present invention is ≦2 weight-%, preferably ≦1 weight-%, more preferably ≦0.5 weight-%, based on the total weight of the active ingredients contained in the composition. That means that beside lycopene and genistein or epigallocatechin gallate and optionally resveratrol, vitamin E, PUFAs, Vitamin D2, vitamin D3 (derivatives) and vitamin C no other active ingredients are present in the compositions used.

### Lycopene

The term “lycopene” includes all-E and Z-stereoisomers. Alternatively a tomato extract which contains high amounts of lycopene could also be used.

### Genistein

Genistein aglycone (4′,5,7-trihydroxyisoflavone) and/or one or more derivatives thereof (genistein glucosides, genistein sulfates, genistein glucuronides);

### Epigallocatechin Gallate

The term “epigallocatechin gallate” (EGCG) encompasses also green tea extracts containing (−)-EGCG as well as (−)-EGCG derivatives such as pharmaceutically acceptable salts.

An especially suitable (−)-EGCG is e.g. Teavigo (a green tea extract containing ≧94% of EGCG), commercially available from DSM Nutritional Products Ltd, Kaiseraugst, Switzerland, as well as Teavigo TG (Tablet Grade) (a green tea extract containing ca. 88% of EGCG admixed with ca. 3% of pectin).

A preferred alternative for (−)-epigallocatechin gallate is a green tea fraction comprising at least 91.7 weight-% of (−)-epigallocatechin gallate (EGCG) and at most 1.43 weight-% of caffeine, especially a green tea fraction comprising from 91.7 to 97.13 weight-% of EGCG, from 0 to 3.15 weight-% of epicatechin (EC), from 0 to 3.1 weight-% of catechin, from 0.2 to 1.52 weight-% of gallocatechin gallate (GCG), from 0.38 to 4.62 weight-% of epicatechin gallate (ECG) and from 0 to 1.43 weight-% of caffeine.

### Vitamin E

The term vitamin E as used herein includes racemic vitamin E (D,L-α-tocopherol) or natural vitamin E, as well as derivatives thereof which have biological vitamin E activity, e.g. carboxylic acid esters, such as vitamin E acetate, propionate, butyrate or succinate.

### Resveratrol

The term resveratrol as used herein includes resveratrol (cis-3,4′,5-trihydroxystilbene and/or trans-3,4′,5-trihydroxystilbene) and/or derivatives thereof which have biological resveratrol activity, e.g. resveratrol glucosides, resveratrol sulfates, resveratrol glucuronides.

### Polyunsaturated Fatty Acids (PUFAs)

The term “PUFAs” encompasses also suitable derivatives such as the ethyl esters of these acids as well as their mono-, di- and tri-glycerides. Triglycerides of n-3 polyunsaturated fatty acids are especially preferred. Hereby mostly 3 different n-3 polyunsaturated fatty acids are esterified with the glycerin. These triglycerides may also contain partly saturated fatty acids. Examples of such n-3 polyunsaturated acids (PUFAs) are eicosapenta-5,8,11,14,17-enoic acid (EPA) and docosahexa-4,7,10,13,16,19-enoic acid (DHA). In one embodiment of the present invention triglycerides are used, whereby 30% of the fatty acid part are n-3 fatty acids and of these 25% are long-chain polyunsaturated fatty acids. In a further embodiment commercially available ROPUFA® ‘30’ n-3 Food Oil (DSM Nutritional Products Ltd, Kaiseraugst, Switzerland) is used.

Alternatively other polyunsaturated fatty acids (omega-3 fatty acids; omega-6 fatty acids) and/or their derivatives may be used.

For the primary and secondary prevention and coadjuvant treatment of prostate cancer including PIN in accordance with the present invention lycopene is administered to the subject in need of such treatment, i.e. humans or pets in an amount of from 0.0005 mg/kg body weight to 5 mg/kg body weight per day. The daily dosage of genistein administered together with lycopene to the subject in need of such treatment, i.e. humans or pets is from 0.17 mg/kg body weight to 1.7 mg/kg body weight. The daily dosage of epigallocatechin gallate administered together with lycopene to the subject in need of such treatment, i.e. humans or pets is from 0.8 mg/kg body weight to 5 mg/kg body weight.

The genistein or epigallocatechin gallate may be administered simultaneously with the lycopene or in advance or afterwards.

“Humans” in the context of the present invention are male humans, especially in the age of from 40 to 80 years. They are the preferred subjects to which the compositions of the present invention are administered.

“Pet animals” in the context of the present invention encompass e.g. cats and dogs; preferably the pets are (male) dogs.

When vitamin E or derivatives thereof is co-administered the daily dosage is from 0.1 mg/kg body weight to 15 mg/kg body weight, based on tocopherol. When resveratrol or a derivative thereof is co-administered the daily dosage is from 0.08 mg/kg body weight to 1.7 mg/lkg body weight, based on resveratrol. When polyunsaturated fatty acid (derivative)s are co-administered the daily dosage is from 1 mg/kg body weight to 50 mg/kg body weight, based on the polyunsaturated fatty acids.

Lycopene and genistein or epigallocatechin gallate may be provided as the active ingredients in compositions, preferably for enteral application, which may be solid or liquid galenical formulations, dietary compositions or animal feed compositions. Examples of solid galenical formulations are tablets, capsules (e.g. hard or soft shell gelatin capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatin, gum arabic, lactose, starch, magnesium stearate, talc, vegetable oils, and the like. Additionally, additives such as flavouring agents, preservatives, stabilizers, emulsifying agents, buffers and the like may be added in accordance with accepted practices of pharmaceutical compounding. They may also be used in dietary compositions which may be a food, a food premix or a fortified food or a beverage. While the individual active ingredients are suitably administered in a single composition they may also be administered in individual dosage units.

Particularly preferred is the administration of the following amounts of the active ingredients:

Lycopene, in a concentration so that the daily consumption by a human adult is in the range of from 0.25 mg/day to 50 mg/day, preferably from 1 mg/day to 30 mg/day; and

Genistein, in a concentration so that the daily consumption by a human adult is in the range of from 10 mg/day to 100 mg/day; or

Epigallocatechin gallate (EGCG), in a concentration so that the daily consumption by a human adult is in the range of from 50 mg/day to 300 mg/day; and/or

Vitamin E or its derivative, in a concentration so that the daily consumption by a human adult is in the range of from 15 mg/day to 600 mg/day; and/or

Resveratrol or its derivative, in a concentration so that the daily consumption by a human adult is in the range of from 5 mg/day to 100 mg/day; and/or

PUFAs are in a concentration so that the daily consumption by a human adult is in the range of from 100 mg/day to 4000 mg/day, preferably from 100 mg/day to 1000 mg/day.

If Vitamin C or its derivative are administered, it is in a concentration so that the daily consumption by a human adult is in the range of from 50 mg/day to 1000 mg/day.

Vitamin D2 or vitamin D3 (derivative) may be co-administered within the following dosage ranges:

| | |
|---|---|
| Vitamin D2 (Ergocalciferol) | 0.1 ng/kg to 10 μg/kg |
| Vitamin D3 (Cholecalciferol) | 0.1 ng/kg to 10 μg/kg |
| 1α,25-Dihydroxyvitamin D3 | 0.1 ng/kg to 0.5 μg/kg |
| 25-Hydroxyvitamin D3 | 0.1 ng/kg to 10 μg/kg |
| 1α,24R,25-Trihydroxyvitamin D3 | 0.1 ng/kg to 0.5 μg/kg |

Typical examples of galenical formulations for use in accordance with the present invention are given below. The Examples are for the purpose of illustrating the invention and are not intended to limit the scope of the invention in any way.

### EXAMPLES

### Example 1

A capsule for the coadjuvant treatment of prostate carcinoma is formulated to contain 5 mg of lycopene, 15 mg of genistein, and optionally 200 mg of vitamin E, 10 mg of resveratrol, and 200 mg of PUFAs. The daily dose corresponds to two such capsules. After 3 weeks of daily supplementation of a man aged 68 years with elevated PSA levels, the plasma PSA value drops. The PSA plasma level is the standard marker for prostate cancer screening and is indicative of prostate size and/or prostatitis. Especially PSA velocity, i.e. the speed by which PSA levels increase, is a strong indication of an emerging prostate carcinoma.

### Example 2

A patient weighing 70 kg and receiving conventional prostate carcinoma therapy is administered, for the duration of the carcinoma therapy, 10 mg of lycopene and 30 mg of genistein per day in a single dosage unit or in individual dosage units of the components. The daily dose corresponds to two such capsules. After 3 weeks of daily supplementation, the plasma PSA value is reduced.

### Example 3

A patient weighing 70 kg and receiving conventional prostate carcinoma therapy is administered, for the duration of the carcinoma therapy, 10 mg of lycopene and 300 mg of epigallocatechin gallate per day in a single dosage unit or in individual dosage units of the components. After 3 weeks of daily supplementation, the plasma PSA value drops.

### Example 4

A patient weighing 70 kg and receiving conventional prostate carcinoma therapy is administered, for the duration of carcinoma therapy, 10 mg of lycopene, 30 mg of genistein, 200 mg of vitamin E, and 37.5 mg of resveratrol per day in a single dosage unit or in individual dosage units of the components. After 3 weeks of daily supplementation, the plasma PSA value is decreased.

1. The use of lycopene and genistein or lycopene and epigallocatechin gallate for the manufacture of a composition for the primary and secondary prevention of prostate carcinoma and coadjuvant treatment thereof.

2. The use as in claim 1 of lycopene and genistein or lycopene and epigallocatechin gallate in combination with at least one of the compounds selected from the group consisting of vitamin E, resveratrol and polyunsaturated fatty acid (derivative)s.

3. The use as in claim 1 of lycopene and genistein or lycopene and epigallocatechin gallate in combination with vitamin E and/or resveratrol.

4. The use as in claim 1, wherein the following compounds are essentially absent in the composition: astaxanthin, β-carotene, β-cryptoxanthin, lutein, quercetin, myricetin, rhizoxin, palmitoyl rhizoxin, silymarin, silybin or equimolar amounts of derivatives, isosilybin or equimolar amounts of derivatives, silydianin or equimolar amounts of derivatives, silychristin or equimolar amounts of derivatives, all-trans retinol, all-trans retinyl acetate, all-trans retinol palmitate, zeaxanthin, apigenin, carnosic acid, carnosol, depudecin, eponemycin, dihydroeponemycin, epoxomicin, ergosterol, fisetin, fumagillin, lactacystin, luteolin, motuporamine C, ovalicin, radicicol, curcumin or equimolar amounts of derivatives, squalamine, isoliquiritin, isoliquiritigenin, shark cartilage extract, glucosinolate derivatives: methylsulfinylalkyl glucosinolates (1-methylsulfinylmethyl glucosinolate, 2-methylsulfinylethyl glucosinolate, 3-methylsulfinylpropyl glucosinolate (glucoiberin), 4-methylsulfinylbutyl glucosinolate (glucoraphanin), 5-methylsulfinylpentyl glucosinolate (glucoalysin), 6-methylsulfinylhexyl glucosinolate, 7-methylsulfinylheptyl glucosinolate, 8-methylsulfinyloctyl glucosinolate, 9-methylsulfinylnonyl glucosinolate, 10-methyl-sulfinyldodecyl glucosinolate) or allyl glucosinolate (sinigrin) or indol-3-ylmethyl glucosinolate (glucobrassicin) or derivatives thereof, (N-methoxyindol-3-ylmethyl glucosinolate (neoglucobrassicin), 4-hydroxyindol-3-ylmethyl glucosinolate (4-OH glucobrassicin), 4-methoxyindol-3-ylmethyl glucosinolate (4-CH3O glucobrassicin)) or phenylethyl glucosinolate (gluconasturtiin) or 3-butenyl glucosinolate (gluconapin)), isothiocyanate derivatives: methylsulfinylalkyl isothiocyanates (1-methylsulfinylmethyl isothiocyanate, 2-methylsulfinylethyl isothiocyanate, 3-methylsulfinylpropyl isothiocyanate, 4-methylsulfinylbutyl isothiocyanate (sulforaphane), 5-methylsulfinylpentyl isothiocyanate, 6-methylsulfinylhexyl isothiocyanate (6-HITC), 7-methylsulfinylheptyl isothiocyanate, 8-methylsulfinyloctyl isothiocyanate, 9-methylsulfinylnonyl-isothiocyanate, 10-methylsulfinyldodecyl isothiocyanate) or allyl isothiocyanate, indol-3-yl-methylisothiocyanate, N-methoxy indol-3-ylmethylisothiocyanate, 4-hydroxy indol-3-ylmethylisothiocyanate, 4-methoxy indol-3-ylmethylisothiocyanate, 3-indolmethanol, phenylethyl isothiocyanate (PEITC), and 3-butenyl isothiocyanate.

5. The use as in claim 1, wherein the composition is a solid or liquid galenical formulation, a dietary composition or an animal feed composition.

6. The use as in claim 5 wherein a daily dosage unit of said solid galenical formulation contains from 5 mg to 30 mg of lycopene and from 10 to 100 mg of genistein or from 50 to 300 mg of epigallocatechin gallate.

7. The use as in claim 5 wherein said liquid galenical formulation contains from 0.1 mg to 100 mg of lycopene and from 1 to 100 mg of genistein or from 20 to 300 mg of epigallocatechin gallate per dose.

8. The use as in claim 5 wherein said dietary composition or animal feed composition contains from 0.025 mg to 10 mg of lycopene and from 1 to 100 mg of genistein or from 20 to 300 mg of epigallocatechin gallate per serving size.

9. The use as in claim 6 wherein a daily dosage unit of said solid galenical formulation further contains from 15 mg to 500 mg of vitamin E.

10. The use as in claim 7 wherein said liquid galenical formulation further contains from 10 mg to 300 mg of vitamin E per dose.

11. The use as in claim 8 wherein said dietary composition or animal feed composition further contains from 1.5 mg to 30 mg of vitamin E per serving size.

12. The use as in claim 6 wherein a dosage unit of said solid galenical formulation further contains from 50 mg to 500 mg of vitamin C.

13. The use as in claim 7 wherein said liquid galenical formulation further contains from 50 mg to 100 mg of vitamin C per dose.

14. The use as in claim 8 wherein said dietary composition or animal feed composition further contains from 5 mg to 50 mg of vitamin C per serving size.

15. The use as in claim 1, in the manufacture of a solid galenical formulation for the coadjuvant treatment of prostate carcinoma containing lycopene and genistein or epigallocatechin gallate in combination with vitamin E, resveratrol, and polyunsaturated fatty acids in one dosage unit.

16. A solid galenical formulation containing per dosage unit (with 2 dosage units per day) 5 mg of lycopene, 15 mg of genistein or 150 mg of epigallocatechin gallate, and optionally 200 mg of vitamin E, 10 mg of resveratrol, and 200 mg of polyunsaturated fatty acids.

17. A method of preventing or treating prostate carcinoma which comprises administering to a subject in need of such treatment for therapy or prophylaxis an effective amount of lycopene and genistein or lycopene and epigallocatechin gallate.

18. The method as in claim 17, wherein from 5 mg to 30 mg of lycopene and from 10 to 100 mg of genistein or from 50 to 300 mg of epigallocatechin gallate are administered per day to a human adult.

19. The method as in claim 17, wherein, additionally, from 15 mg to 600 mg of vitamin E are administered per day to a human adult.

20. The method as in claim 17, wherein, additionally, from 5 to 100 mg of resveratrol are administered per day to a human adult.

21. A method for the coadjuvant treatment of prostate carcinoma which comprises administering to a human adult lycopene and genistein or epigallocatechin gallate in combination with vitamin E, resveratrol and polyunsaturated fatty acids.

22. The method as in claim 21, wherein 10 mg of lycopene, 30 mg of genistein or 300 mg of epigallocatechin gallate, from 20 to 37.5 mg of resveratrol, from 200 to 400 mg of vitamin E, and 400 mg of polyunsaturated fatty acids are administered to a human adult per day.

23. A method for the coadjuvant treatment of prostate carcinoma which comprises administering to a human adult lycopene and genistein or lycopene and epigallocatechin gallate in combination with at least one of the compounds selected from the group consisting of vitamin E, resveratrol and polyunsaturated fatty acid (derivative)s.

24. Use of lycopene and genistein or lycopene and epigallocatechin gallate for the manufacture of a composition for reducing/decreasing the PSA plasma level in the blood of subjects to whom either said composition comprising lycopene and genistein is administered or to whom either said composition comprising lycopene and epigallocatechin gallate is administered.

25. A method of reducing/decreasing the PSA plasma level in the blood of a subject which comprises administering to said subject in need of such treatment an effective amount of lycopene and genistein or an effective amount of lycopene and epigallocatechin gallate.

26. The use of lycopene and genistein for the manufacture of a composition for the primary and secondary prevention of prostate carcinoma and/or for the coadjuvant treatment thereof.

27. The use of lycopene and epigallocatechin gallate for the manufacture of a composition for the primary and secondary prevention of prostate carcinoma and/or for the coadjuvant treatment thereof.

28. A method of preventing or treating prostate carcinoma which comprises administering to a subject in need of such treatment for therapy or prophylaxis an effective amount of lycopene and genistein.

29. A method of preventing or treating prostate carcinoma which comprises administering to a subject in need of such treatment for therapy or prophylaxis an effective amount of lycopene and epigallocatechin gallate.

30. A method for the coadjuvant treatment of prostate carcinoma which comprises administering to a human adult lycopene and genistein in combination with at least one of the compounds selected from the group consisting of vitamin E, resveratrol and polyunsaturated fatty acid (derivative)s.

31. A method for the coadjuvant treatment of prostate carcinoma which comprises administering to a human adult lycopene and epigallocatechin gallate in combination with at least one of the compounds selected from the group consisting of vitamin E, resveratrol and polyunsaturated fatty acid (derivative)s.
